(19) United States
(12) Patent Application Publication
(10) Pub. No.: US 2026/0151480 A1
(43) Pub. Date: Jun. 4, 2026

(54) METHOD AND KIT FOR TREATING MICROSATELLITE-STABLE SOLID TUMORS

(71) Applicant: GOLDEN HEALTH (GUANGDONG) BIOTECHNOLOGY CO., LTD., Foshan (CN)

(72) Inventors: Jinlin ZHOU, Foshan (CN); Liwei LIN, Foshan (CN); Yanfeng HU, Foshan (CN); Yujing LU, Foshan (CN); Zhishan ZHOU, Foshan (CN); Xiang YANG, Foshan (CN); Baohua HUANG, Foshan (CN); Huiling LI, Foshan (CN)

(73) Assignee: GOLDEN HEALTH (GUANGDONG) BIOTECHNOLOGY CO., LTD., Foshan (CN)

(21) Appl. No.: 19/461,806

(22) Filed: Jan. 28, 2026

Related U.S. Application Data
(63) Continuation-in-part of application No. PCT/CN2023/138980, filed on Dec. 15, 2023.

(30) Foreign Application Priority Data
Jul. 28, 2023 (CN) 202310947489.2

(51) Int. Cl. A61K 39/395; A61K 31/7048; A61K 40/11; A61K 40/31; A61K 40/42; A61P 35/00

(52) U.S. Cl. CPC A61K 39/3955; A61K 31/7048; A61K 40/11; A61K 40/31; A61K 40/4211; A61P 35/00

(57) ABSTRACT

A method for treating a microsatellite-stable (MSS) solid tumor is provided. The method includes administering a therapeutically effective amount of icariside I to a subject suffering from the MSS solid tumor. The method further includes administering a combination anti-cancer active agent to the subject, the combination anti-cancer active agent includes an immunotherapeutic agent (for example, a PD-1/PD-L1 inhibitor and/or a CAR-T therapy product). A use of the icariside I in the preparation of a drug for treating or preventing the MSS solid tumor, particularly a MSS gastric cancer, a MSS intestinal cancer, a MSS lung cancer, a MSS pancreatic cancer, and a MSS hepatocellular carcinoma, and a kit including the same are further provided. A use of a combination of the icariside I and the immunotherapeutic agent in the preparation of a drug for treating or preventing the MSS solid tumor.

### CROSS-REFERENCE TO RELATED APPLICATIONS

This application is a continuation-in-part application of International Application No. PCT/CN/2023/138980, filed on Dec. 15, 2023, which claims priority to Chinese Patent Application No. 202310947489.2, filed on Jul. 28, 2023, the entire contents of each of which are hereby incorporated by reference.

### TECHNICAL FIELD

The present disclosure generally relates to the field of anti-tumor technology, and in particular to a method and a kit for treating a microsatellite-stable solid tumor.

### BACKGROUND

In 2020, proportions of newly diagnosed cancer cases in China for the following cancers were as follows: lung cancer, 17.9%; intestinal cancer, 12.2%; gastric cancer, 10.5%; hepatocellular carcinoma, 9%; and pancreatic cancer, 2.7%. These five cancers together accounted for 52.3% of all newly diagnosed cancer cases.

Microsatellite (MS) refers to short tandem repeat sequences dispersed throughout an entire human genome. Microsatellite instability (MSI) refers to a phenomenon in which a length of short repeat DNA sequences is altered due to insertion or deletion mutations during DNA replication, which is often caused by defects in mismatch repair (MMR) function. MSI may be classified into three categories based on degree of instability: microsatellite instability-high (MSI-high, MSI-H), microsatellite instability-low (MSI-low, MSI-L), and microsatellite-stable (MSS). Correspondingly, from a perspective of MMR gene function, the MSI may be classified as deficient mismatch repair (dMMR) and proficient mismatch repair (pMMR). The MSI-H corresponds to the dMMR, while the MSI-L and the MSS correspond to the pMMR. Since MSI-L tumors and the MSS tumors exhibit similar clinical characteristics, the MSI-L tumors and the MSS tumors are generally grouped together and collectively referred to as the MSS in clinical subtype analysis. Typically, in clinical practice, microsatellite instability-high tumors are referred to as MSI-H/dMMR tumors, and microsatellite stability-high tumors and microsatellite instability-low tumors are collectively referred to as MSS/pMMR tumors. In the present disclosure, for convenience, MSS is also used to replace MSS/pMMR, that is, the term “MSS” is used to describe the microsatellite stability-high tumors and the microsatellite instability-low tumors.

For most patients with solid tumors, PD-1/PD-L1 inhibitors are only effective in 20%-30% of the patients. Among these responsive patients, 10%-15% of the patients subsequently develop drug resistance. Incidence rates of MSI-H in the gastric cancer, the intestinal cancer, the hepatocellular carcinoma, the pancreatic cancer, and the lung cancer are approximately 22%, 15%, 18%, 22%, and 5%, respectively. The remaining MSS do not respond to the PD-1/PD-L1 inhibitors.

Chimeric antigen receptor T-cell immunotherapy (CAR-T therapy) is another important approach for tumor treatment. However, CAR-T therapy is most effective against hematologic cancers but shows limited efficacy against the solid tumors.

In the present disclosure, the term “MSS” is used to describe the above-mentioned tumor or cancer subtypes that are insensitive to immunotherapy.

To overcome a challenge of poor efficacy of the immunotherapy, neoadjuvant treatment strategies combining chemotherapy and immunotherapy have been explored. Potential mechanisms of synergistic effect include immunogenic tumor cell death, anti-angiogenesis, selective depletion of myeloid-derived suppressor cells, or the like. However, unfortunately, although chemotherapy is effective, the chemotherapy largely lacks a bystander killing effect on tumors. At tolerable doses, the remaining malignant cells are most likely to escape and develop resistance. Simultaneously, high-dose chemotherapy causes immunosuppression. Therefore, there remains an urgent clinical need for a safe, low-toxicity, and highly effective therapeutic drug for treating MSS solid tumors.

### SUMMARY

One or more embodiments of the present disclosure provide a method for treating a microsatellite-stable (MSS) solid tumor. The method includes administering a therapeutically effective amount of icariside I to a subject suffering from the MSS solid tumor.

In some embodiments, the MSS solid tumor is selected from the group consisting of a microsatellite-stable gastric cancer, a microsatellite-stable intestinal cancer, a microsatellite-stable lung cancer, a microsatellite-stable pancreatic cancer, and a microsatellite-stable hepatocellular carcinoma.

In some embodiments, the icariside I is in a form of a tablet, a capsule, a granule, a softgel, a drop pill, a syrup, an injection, or a loaded nanocomposite.

In some embodiments, the method further includes administering a combination anti-cancer active agent to the subject.

In some embodiments, the combination anti-cancer active agent includes an immunotherapeutic agent.

In some embodiments, the immunotherapeutic agent includes a PD-1/PD-L1 inhibitor or a CAR-T therapy product.

In some embodiments, the PD-1/PD-L1 inhibitor includes at least one of nivolumab, sintilimab, camrelizumab, atezolizumab, or pembrolizumab.

In some embodiments the CAR-T therapy product includes at least one of axicabtagene ciloleucel or relmacabtagene autoleucel.

In some embodiments, the therapeutically effective amount of the icariside I is 10-120 mg per kg body mass of the subject (mg/kg).

In some embodiments, the therapeutically effective amount of the icariside I is 20-80 mg/kg.

In some embodiments, the method further includes: administering a combination anti-cancer active agent including an immunotherapeutic agent to the subject, and the therapeutically effective amount of the icariside I is 30 mg/kg.

In some embodiments, the administering the icariside I includes: administering the icariside I once daily by intragastric administration or oral administration for 7 consecutive days.

In some embodiments, the method further includes: injecting an immunotherapeutic agent on day 1, day 4, and day 7.

In some embodiments, the immunotherapeutic agent is nivolumab.

In some embodiments, the immunotherapeutic agent is axicabtagene ciloleucel.

In some embodiments, the method further includes: administering nivolumab and axicabtagene ciloleucel to the subject.

One or more embodiments of the present disclosure provide a kit for treating a MSS solid tumor, and the kit includes icariside I.

In some embodiments, the kit further includes an immunotherapeutic agent.

In some embodiments, the immunotherapeutic agent includes at least one of a PD-1/PD-L1 inhibitor or a CAR-T therapy product.

In some embodiments, the icariside I and the immunotherapeutic agent are individually packaged or co-packaged.

In some embodiments, the MSS solid tumor treated by the kit is selected from the group consisting of a microsatellite-stable gastric cancer, a microsatellite-stable intestinal cancer, a microsatellite-stable lung cancer, a microsatellite-stable pancreatic cancer, and a microsatellite-stable hepatocellular carcinoma.

### BRIEF DESCRIPTION OF THE DRAWINGS

To more clearly illustrate technical solutions of some embodiments of the present disclosure, some embodiments of the present disclosure are described below with reference to the drawings. It is obvious that the drawings in the following description relate only to some implementations of the present disclosure.

FIG. 1 is a schematic diagram illustrating an effect of icariside I on cell viability of gastric cancer cells (CCK-8 cells) at different doses in Example 1.

FIG. 2 is a schematic diagram illustrating a result of a live/dead staining assay of icariside I on gastric cancer cells in Example 1.

FIG. 3 is a schematic diagram illustrating reactive oxygen species levels in gastric cancer cells after intervention with icariside I in Example 2.

FIG. 4 is an ex vivo photograph of gastric cancer tumor tissues from mice after oral administration with icariside I in Example 3.

FIG. 5 is a schematic diagram illustrating a tumor volume of gastric cancer after oral administration with icariside I in Example 3.

FIG. 6 is a flow cytometry analysis plot of mature dendritic cells (DCs) in lymph node tissues of mice after oral administration with icariside I in Example 4.

FIG. 7 is a quantitative analysis statistical chart of mature dendritic cells (DCs) in lymph node tissues of mice after oral administration with icariside I in Example 4.

FIGS. 8A and 8B are schematic diagrams illustrating therapeutic efficacy of icariside I combined with anti-programmed cell death protein 1 (A-PD-1) intervention on microsatellite-stable gastric cancer model mice in Example 5, where FIG. 8A illustrates tumor weight in different groups, and FIG. 8B illustrates tumor growth curves over days after treatment.

FIG. 9 is an ex vivo photograph of microsatellite-stable intestinal cancer tumor tissues after intervention with icariside I alone or in combination with A-PD-1 in Example 6.

FIG. 10 is a schematic diagram illustrating a tumor volume of microsatellite-stable intestinal cancer after intervention with icariside I alone or in combination with A-PD-1 in Example 6.

FIG. 11 is a schematic diagram illustrating a tumor volume of microsatellite-stable lung cancer after intervention with icariside I alone or in combination with A-PD-1 in Example 7.

FIG. 12 is a schematic diagram illustrating a tumor volume of microsatellite-stable hepatocellular carcinoma after intervention with icariside I alone or in combination with A-PD-1 in Example 8.

FIG. 13 is a schematic diagram illustrating a tumor volume of microsatellite-stable pancreatic cancer after intervention with icariside I alone or in combination with A-PD-1 in Example 9.

FIG. 14 is a schematic diagram illustrating a tumor volume of microsatellite-stable tumor after intervention with icariside I in Example 10.

FIG. 15 is a schematic diagram illustrating a tumor volume of microsatellite-stable tumor after intervention with icariside I alone or in combination with a chimeric antigen receptor T cell (CAR-T) therapy product in Example 11.

FIG. 16 is a schematic diagram illustrating a tumor volume of microsatellite-stable tumor after intervention with icariside I alone or in combination with A-PD-1 in Example 12.

FIG. 17 is a schematic diagram illustrating a tumor volume of microsatellite-stable tumor after intervention with icariside I alone or in combination with A-PD-1 and a CAR-T therapy product in Example 13.

### DETAILED DESCRIPTION

To make the technical problems, technical solutions, and beneficial effects of the present disclosure clearer and more comprehensible, the technical solutions of the present disclosure are described clearly and completely below in combination with embodiments. It should be understood that the specific embodiments described herein are merely for explaining the present disclosure and are not intended to limit the present disclosure.

One or more embodiments of the present disclosure provide a method for treating a microsatellite-stable (MSS) solid tumor. The method includes administering a therapeutically effective amount of icariside I to a subject suffering from the MSS solid tumor.

The MSS solid tumor refers to a solid tumor characterized by proficient mismatch repair (pMMR). In clinical practice, microsatellite stability-high and microsatellite instability-low (MSI-low, MSI-L) are collectively referred to as MSS.

In some embodiments, the MSS solid tumor is selected from the group consisting of a MSS gastric cancer, a MSS intestinal cancer, a MSS lung cancer, a MSS pancreatic cancer, and a MSS hepatocellular carcinoma.

For the above five cancer types (the MSS gastric cancer, the MSS intestinal cancer, the MSS lung cancer, the MSS pancreatic cancer, and the MSS hepatocellular carcinoma), the MSS subtypes are usually insensitive to conventional immunotherapy in the clinical practice. Administration of icariside I can effectively activate immune response in the microenvironment of these specific cancer types, providing a new clinical treatment pathway.

The subject refers to an object receiving a drug, a kit, or a treatment manner. In some embodiments, the subject includes an experimental animal (e.g., a tumor-bearing mouse) and a human.

icariside I refers to a glycosidic natural product derived from Epimedium spp., which is an active component of Epimedium medicinal material. A structural formula of the icariside I is as follows:

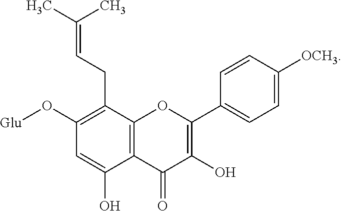

The icariside I may be a naturally isolated product or a same chemical entity synthesized by a chemical or biological method. Preferably, raw material of the icariside I has a purity of not less than 90%. More preferably, preparations with a purity of not less than 95% or higher are used as a pharmaceutically active ingredient. The icariside I intended for formulation may be prepared by conventional refining, crystallization, or chromatographic purification processes.

In some embodiments, the icariside I may be used for treatment or prevention of the MSS solid tumor.

In some embodiments, the icariside I is in a form of a tablet, a capsule, a granule, a softgel, a drop pill, a syrup, an injection, or a loaded nanocomposite.

The icariside I may be processed with suitable excipients into a specific administration form according to disease treatment needs and drug properties. For example, dosage forms include, but are not limited to, oral solid preparations (e.g., the tablet, the capsule, the granule, the softgel, and the drop pill), oral liquid preparations (e.g., the syrup), injectable preparations (e.g., the injection), and advanced delivery systems (e.g., the loaded nanocomposite).

The loaded nanocomposite refers to a composite formed by loading the icariside I into a nanoscale carrier (e.g., liposome, polymeric micelle, inorganic nanoparticle, or the like) using nanotechnology. The loaded nanocomposite aims to improve solubility, stability, and tumor targeting ability of the icariside I.

By preparing the icariside I as the tablet, the capsule, the granule, the softgel, the drop pill, the syrup, the injection, or the loaded nanocomposite, application forms of the active ingredient in the clinical treatment of the MSS solid tumor are expanded, enabling it to meet requirements of different administration routes and treatment stages. The above diversified dosage forms not only improve administration flexibility and patient compliance, but also can improve onset speed, bioavailability, and targeting ability of the drug through injection or nano-delivery manners, thereby overall enhancing therapeutic effects and reducing potential adverse reactions.

In some embodiments, the method for treating a MSS solid tumor further includes administering a combination anti-cancer active agent to the subject.

The combination anti-cancer active agent refers to another substance having anti-tumor activity that is co-administered with the icariside I to achieve purposes of enhancing efficacy, reducing toxicity, or overcoming drug resistance. The combination anti-cancer active agent may act on a same biological target as the icariside I or may produce a synergistic effect through different mechanisms.

In some embodiments, the combination anti-cancer active agent may include an immune checkpoint inhibitor, an immunomodulatory small molecule or biologic agent, a chemotherapeutic drug, a targeted drug, radiotherapy, cell therapy, an oncolytic virus, and an immunity-enhancing adjuvant approach.

In some embodiments, the combination anti-cancer active agent includes an immunotherapeutic agent.

The immunotherapeutic agent refers to an active pharmaceutical compound that exerts a therapeutic effect by acting on an immune system of the subject. For example, the immunotherapeutic agent includes, but is not limited to, an immune checkpoint inhibitor, an immunostimulatory cytokine, an immunomodulatory small molecule, a tumor vaccine, an engineered cell therapy product, and combinations thereof.

In some embodiments, the immunotherapeutic agent includes at least one of a programmed cell death protein 1/programmed cell death ligand 1 (PD-1/PD-L1) inhibitor or a chimeric antigen receptor T-cell (CAR-T) therapy product.

The PD-1/PD-L1 inhibitor refers to an inhibitor compound capable of enhancing T cell activity and exerting an anti-cancer effect by blocking a PD-1/PD-L1 signaling pathway.

In some embodiments, the PD-1/PD-L1 inhibitor includes at least one of nivolumab, sintilimab, camrelizumab, atezolizumab, or pembrolizumab.

The CAR-T therapy product refers to a therapeutic agent used in chimeric antigen receptor T cell immunotherapy. For example, the CAR-T therapy product includes autologous or allogeneic T cells that are collected in vitro and genetically engineered to express chimeric antigen receptors (CARs), which are used to recognize and kill tumor cells expressing corresponding tumor antigens.

In some embodiments, the CAR-T therapy product includes at least one of axicabtagene ciloleucel or relmacabtagene autoleucel.

The axicabtagene ciloleucel and the relmacabtagene autoleucel (including relatlimab-targeted products and other approved or investigational autologous T-cell immunotherapy products targeting specific antigens such as CD19) are both CAR-T therapeutic preparations.

Combination application of the icariside I and the combination anti-cancer active agent can significantly enhance therapeutic effects on the MSS solid tumor and reduce a clinical dosage of a single drug. Furthermore, while maintaining or improving efficacy, severe adverse reactions caused by chemotherapy or targeted drugs can be alleviated. Through combination of drugs with different mechanisms, occurrence and development of the tumors can be more comprehensively inhibited, and possibilities of tumor escape and recurrence can be reduced.

In some embodiments of the present disclosure, the icariside I can effectively enhance anti-tumor immune therapeutic effects of the immunotherapeutic agent (e.g., the PD-1/PD-L1 monoclonal antibody or the CAR-T therapy product). The icariside I synergistically acts with the immunotherapeutic agent (e.g., the PD-1/PD-L1 inhibitor and/or the CAR-T therapy product) to further improve effects in the treatment of the MSS solid tumor.

The therapeutically effective amount refers to a dose of the active ingredient icariside I that produces a desired therapeutic, inhibitory, or alleviating effect on the MSS solid tumor in the subject.

In some embodiments, the therapeutically effective amount of the icariside I is 10-120 mg per kg body mass of the subject (mg/kg).

In some embodiments, the therapeutically effective amount of the icariside I is 20-80 mg/kg.

In some embodiments, the therapeutically effective amount of the icariside I is 10-20 mg/kg, 20-30 mg/kg, 30-40 mg/kg, 40-50 mg/kg, 50-60 mg/kg, 60-70 mg/kg, 70-80 mg/kg, 40-80 mg/kg, 80-120 mg/kg, or the like.

In some embodiments, the therapeutically effective amount of the icariside I is 20 mg/kg, 30 mg/kg, 40 mg/kg, 60 mg/kg, 80 mg/kg, 100 mg/kg, 120 mg/kg, or the like.

Ranges such as 10-120 mg/kg, 20-80 mg/kg, etc., recorded in some embodiments of the present disclosure, include all specific numerical values and any sub-ranges within the ranges. Those skilled in the art should understand that, although the present disclosure recommends these ranges, fine-tuning of the dosage based on individual circumstances should be considered within an equivalent protection scope of the claims.

Limiting a dosage of the icariside I to a range of 10-120 mg/kg or 20-80 mg/kg has significant technical effects. Within these ranges, the drug can maintain a stable tumor inhibition rate, thereby avoiding a problem of an insignificant tumor inhibition effect that may occur when the dosage is below 10 mg/kg. In addition, the ranges significantly improve medication safety. In the administration experiments, subjects in the 20-80 mg/kg group exhibit normal blood test parameters and organ histopathology, without reaching a toxicity threshold. This preferred dosage interval provides an extremely robust benchmark for clinical combination therapy and has clinical application value and scientific validity.

In some embodiments, a combination anti-cancer active agent including an immunotherapeutic agent is administered to the subject, and the therapeutically effective amount of the icariside I is 30 mg/kg.

In some embodiments of the present disclosure, a combination of 30 mg/kg the icariside I and the immunotherapeutic agent can balance sensitization activity and the safety. At this dosage, the combination of the icariside I and the PD-1/PD-L1 inhibitor can increase the tumor inhibition rate for models such as an MSS gastric cancer, an MSS lung cancer, and an MSS hepatocellular carcinoma from an extremely low level of monotherapy to about 78% (see corresponding content of Example 7), which is significantly better than an effect of a higher dosage monotherapy or a low-dosage combination therapy. This dosage is beneficial for reversing a situation where an MSS tumor does not respond to immunotherapy. Meanwhile, a dosage of 30 mg/kg is far below a toxicity threshold, which can effectively avoid systemic inflammatory reactions or liver and kidney function damage caused by drug superposition during long-term combination therapy.

In some embodiments, administering the icariside I includes: administering the icariside I once daily by intragastric administration or oral administration for 7 consecutive days.

Both the intragastric administration and the oral administration refer to administration manners via a digestive tract that enable the subject to absorb the icariside I through a gastrointestinal tract.

In some embodiments, an administration regimen is once daily by intragastric administration or oral administration for 7 consecutive days. For example, the subject is administered, by the oral administration or the intragastric administration, with the optional therapeutically effective amount (for example, 20-80 mg/kg) described in the present disclosure at a same time each day, for 7 consecutive days, thereby constituting a complete treatment course.

Administering the icariside I according to a regimen of once daily for 7 consecutive days can establish stable and controllable in vivo exposure in a short term, thereby helping to induce a tumor-related immune response and providing a clear time window for combination with the immunotherapeutic agent. This administration regimen facilitates dosage management and tolerance evaluation, which is beneficial for reproducible implementation of preclinical and early clinical trials, thereby improving reliability of efficacy evaluation and accelerating clinical translation of combination therapy strategies.

In some embodiments, the method further includes: injecting the immunotherapeutic agent on day 1, day 4, and day 7. In some embodiments, the immunotherapeutic agent is nivolumab. In some embodiments, the immunotherapeutic agent is axicabtagene ciloleucel. In some embodiments, the method further includes: administering nivolumab and axicabtagene ciloleucel to the subject.

Administration is performed once on day 1, day 4, and day 7 of a treatment cycle, and this schedule may form a synergistic administration window with concurrent oral administration or intragastric administration (for example, administration of the icariside I once daily for 7 consecutive days). The administration and the oral administration or intragastric administration may overlap in time or be carried out in coordination according to a predetermined timing sequence.

In some embodiments, the subject receives the injection of the selected combination anti-cancer active agent on day 1, day 4, and day 7, and simultaneously receives the oral administration or intragastric administration of the icariside I (for example, 30 mg/kg as an exemplary dosage) daily from day 1 to day 7.

Scheduling injection of the immunotherapeutic agent on day 1, day 4, and day 7 is intended to synchronously or sequentially amplify an immune response within a time window during which the icariside I induces tumor stress or promotes tumor antigen release. Oral administration for 7 consecutive days may be performed in advance, or partially overlap with the injection, thereby intervening in a tumor immune microenvironment at a plurality of time points and increasing a cumulative effect of immune activation and tumor cell clearance. The timing sequence is provided as an exemplary embodiment, and specific time points, frequencies, and administration sequence may be optimized based on pharmacokinetic, pharmacodynamic, and safety data.

Injection of the combination anti-cancer active agent on day 1, day 4, and day 7 utilizes a preheating effect of the icariside I on an immune microenvironment. In a context of continuous administration, pulsed administration on day 1, day 4, and day 7 is beneficial for activating infiltration and killing activity of T cells. Such a regular and intermittent administration regimen helps reduce a medication burden on the subject and enables effective monitoring of occurrence of immune-related adverse reactions, thereby balancing high efficacy with high safety and offering clinical operational value.

In some embodiments of the present disclosure, the icariside I can effectively increase reactive oxygen species level in MSS solid tumor cells and significantly inhibit growth of the MSS solid tumor cells, thereby exhibiting an excellent therapeutic effect on the MSS solid tumor. For example, an in vivo inhibition rate of the icariside I against MSS tumors such as the MSS gastric cancer, the MSS intestinal cancer, and the MSS lung cancer can reach 70%-80%. Moreover, the icariside I is derived from a natural medicinal material, providing a safe and effective new therapeutic option for clinical treatment of MSS solid tumors.

One or more embodiments of the present disclosure provide a kit for treating a MSS solid tumor, and the kit includes icariside I.

The kit refers to a pharmaceutical product form that integrates a plurality of components, devices, or instructional materials for treating a specific disease within a single package.

In some embodiments, the kit includes an active ingredient icariside I, which may exist in a single dosage form (such as a tablet, a capsule, or an injection).

In some embodiments of the present disclosure, components of the kit are not limited to the icariside I, and may further include a diluent, a solvent, an administration device (e.g., a syringe and an infusion tube), or an auxiliary diagnostic reagent for monitoring a treatment effect according to clinical needs.

In some embodiments, the kit further includes an immunotherapeutic agent. More descriptions of the immunotherapeutic agent may be found in corresponding descriptions above.

A combination kit refers to an integrated packaging system designed to implement a combination administration regimen; and the kit simultaneously includes the active ingredient icariside I and at least one immunotherapeutic agent.

In some embodiments, the immunotherapeutic agent includes at least one of a PD-1/PD-L1 inhibitor or a CAR-T therapy product. More descriptions of the PD-1/PD-L1 inhibitor or the CAR-T therapy product may be found in corresponding descriptions above.

In some embodiments, the icariside I and the immunotherapeutic agent are individually packaged or co-packaged.

The individual packaging refers to placing an icariside I preparation and an immunotherapeutic agent preparation in their respective independent inner packaging containers, such as different vials, aluminum foil plates, or prefilled syringes. Physical isolation can ensure that physicochemical properties of components are not interfered with.

The co-packaging refers to integrating the icariside I and the immunotherapeutic agent, which have been pre-formulated or specifically processed, in a same administration unit or a mixed container, or achieving co-loading of the icariside I and the immunotherapeutic agent through forms such as a dual-chamber bag or a composite preparation.

In the therapeutic methods provided in the present disclosure, corresponding operations are adopted for use of the kit according to a packaging form of the kit. If the individual packaging is adopted, a treatment process manifests as sequential administration: the individually packaged icariside I (e.g., the tablets) is first taken from the kit for daily oral administration, and the individually packaged immunotherapeutic agent (e.g., the PD-1 inhibitor) is taken for injection on specific days, thereby utilizing a preheating effect of the icariside I on a tumor microenvironment. If the co-packaging is adopted, the treatment process tends more towards synchronous administration, that is, simultaneously administering a compound preparation or a nanocomposite containing the icariside I and an immune component through a single operation, thereby ensuring that the icariside I and the immune component have consistent biodistribution in tumor tissue.

By supporting the individual packaging or the co-packaging of the icariside I and the immunotherapeutic agent, the co-packaging facilitates precise administration according to a timing sequence and reduces error risk, while the individual packaging facilitates decentralized distribution and targeted adjustment of a treatment regimen, which helps ensure medication safety.

Embodiments of the present disclosure provide a combination kit including the icariside I and the immunotherapeutic agent, simplifying a complex combination administration process and helping to improve an effective rate of immunotherapy. Meanwhile, the combination kit not only facilitates logistics distribution and hospital inventory management, but also helps achieve standardization of the treatment process in clinical promotion.

In one or more embodiments of the present disclosure, the kit form can effectively improve chemical stability of the drug, and the reasonable packaging design (such as light-proof and moisture-proof treatment) ensures that activity of the icariside I does not decrease during storage and transportation. This design improves convenience of administration, dosage accuracy, and treatment compliance, accelerates clinical translation, and increases treatment benefits for patients with the MSS solid tumor.

The kit of some embodiments of the present disclosure includes the icariside I. Further, a combination anti-cancer active agent may be added to the kit. The combination anti-cancer active agent may be the immunotherapeutic agent, preferably a PD-1/PD-L1 inhibitor and a CAR-T therapy product. The icariside I may exist in the kit in a form of the individual packaging. Alternatively, the icariside I may exist in the kit in a form of a combination formed with other components. Adding the combination anti-cancer active agent may further improve an effect of the kit for treating the MSS solid tumor. For subjects with a MSS gastric cancer, a MSS intestinal cancer, a MSS lung cancer, a MSS pancreatic cancer, or a MSS hepatocellular carcinoma, the icariside I can effectively enhance an anti-tumor immune therapeutic effect of the immunotherapeutic agent (for example, a PD-1/PD-L1 monoclonal antibody or the CAR-T therapy product).

Some embodiments of the present disclosure provide a use of the icariside I in the preparation of a drug for treating or preventing the MSS solid tumor. In particular, for the MSS solid tumors, especially the MSS gastric cancer, the MSS intestinal cancer, the MSS lung cancer, the MSS pancreatic cancer, and the MSS hepatocellular carcinoma, the icariside I has a good therapeutic or preventive effect.

Some embodiments of the present disclosure further provide a use of a combination of the icariside I and the immunotherapeutic agent (for example, the PD-1/PD-L1 inhibitor and/or the CAR-T therapy product) in the preparation of a drug for treating or preventing the MSS solid tumor. In a particular embodiment, the MSS solid tumor is the MSS gastric cancer, the MSS intestinal cancer, the MSS lung cancer, the MSS pancreatic cancer, or the MSS hepatocellular carcinoma.

Reagents or instruments used without specifying manufacturers are conventional products available through commercial purchase.

In some embodiments of the present disclosure, the icariside I serves as an active ingredient, raw material of which has a purity of preferably above 90%, more preferably above 95%, above 96%, above 97%, above 98%, above 98.5%, above 99%, and above 99.5%. In some embodiments of the present disclosure, a dosage form of the drug includes the tablet, the capsule, the granule, the softgel, the drop pill, the syrup, the injection, or the loaded nanocomposite. In some embodiments of the present disclosure, the drug includes the icariside I as a sole active ingredient. Alternatively, the drug includes a combination of the icariside I and another active pharmaceutical compound (for example, the PD-1/PD-L1 inhibitor and/or the CAR-T therapy product) as the active ingredient. Some embodiments of the present disclosure do not impose special limitations on a preparation method of the drug, which may be prepared using a conventional preparation method for various dosage forms.

### Example 1

### In Vitro Anti-Tumor (Gastric Cancer) Effect of Icariside I at Different Doses

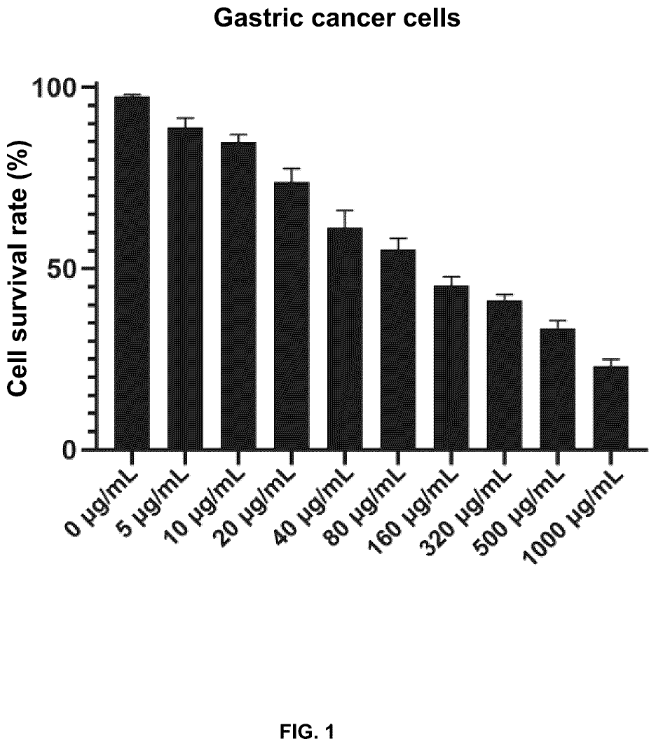

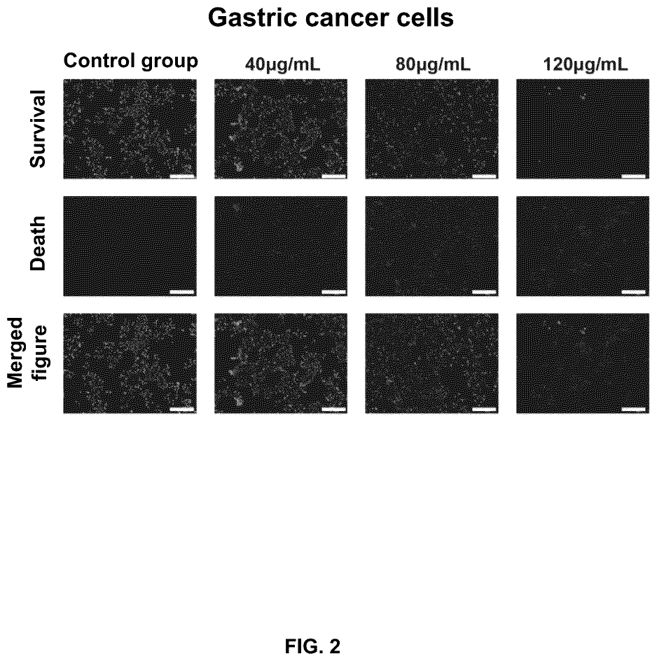

First, mouse forestomach carcinoma (MFC) cells in a logarithmic growth phase were digested with trypsin to obtain a single-cell suspension. The single-cell suspension was plated on a culture plate and cultured in a constant-temperature incubator. After 24 hours, when the cells adhered, different doses of icariside I were added to each well. Subsequently, the culture plate was placed in the incubator for another 24 hours of culture, followed by a CCK-8 cytotoxicity assay and a live/dead cell staining assay, and results are shown in FIG. 1 and FIG. 2.

Experimental results: As can be seen from FIG. 1, the icariside I has a significant inhibitory effect on the growth of MFC cells in vitro and exhibits a dose-effect relationship. An IC50 of the icariside I for the MFC cells is 90 μg/mL. As can be seen from FIG. 2, compared with a blank control group, MFC cells treated with a low-dose group (40 μg/mL) exhibit higher cell viability and lower mortality, indicating an insignificant anti-tumor effect; in contrast, MFC cells treated with a medium-dose group (80 μg/mL) and a high-dose group (120 μg/mL) exhibit a significant reduction in a count of live cells and an increase in dead cells, indicating that the icariside I has a more pronounced tumor cell-killing effect at medium and high doses. Therefore, as can be seen from FIG. 1 and FIG. 2, as a dose increases, the icariside I exhibits a stronger inhibitory effect on the growth of MFC cells in vitro.

### Example 2

Detection of Reactive Oxygen Species in Gastric Cancer Cells after Intervention with Icariside I at Different Doses

For detection of a reactive oxygen species level, MFC cells in a logarithmic growth phase were first digested with 0.25% trypsin to obtain a single-cell suspension. The single-cell suspension was inoculated into a 6-well plate and cultured in a constant-temperature incubator for 24 hours. After the cells adhered, different doses of icariside I were added to each well for treatment. The 6-well plate was then placed back in the incubator for another 24 hours of culture. Then, a 2′,7′-Dichlorodihydrofluorescein diacetate (DCFH-DA) reagent was diluted with serum-free medium at a ratio of 1000:1 and added into the each well. The 6-well plate was placed in the constant-temperature incubator for further incubation. After 20 minutes, the cells were observed under an inverted fluorescence microscope.

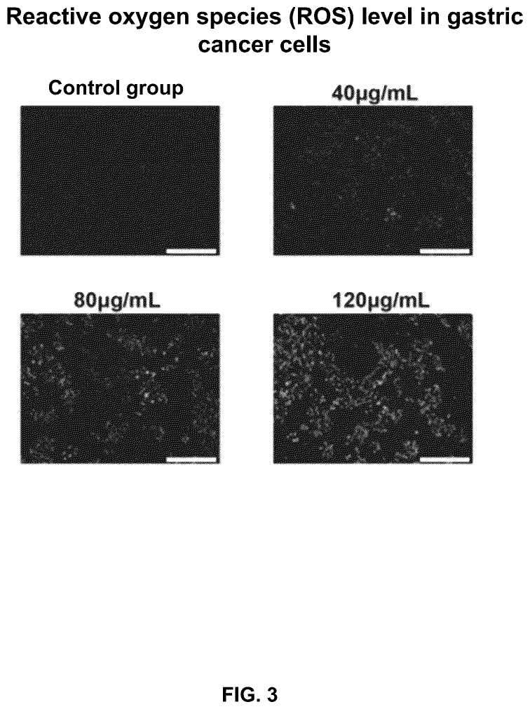

As shown in FIG. 3, the icariside I can induce an increase in reactive oxygen species level in the MFC cells. As the drug dose increases, a reactive oxygen species level in the MFC cells increases accordingly. This indicates that the icariside I can cause an increase in the reactive oxygen species level in the MFC cells in a dose-dependent manner.

### Example 3

Inhibitory Effect on Tumor Growth of MSS Gastric Cancer after Intervention with Icariside I at Different Doses

To elucidate an in vivo therapeutic effect of icariside I, subcutaneous gastric cancer tumor-bearing balb/c mouse models (MSS) were successfully constructed.

Construction of MSS gastric cancer mouse model was as follows:

a. Construction of a Lentivirus-Transfected MFC Cell Line

The MFC cell line in a logarithmic growth phase was selected. The MFC cell line was digested with trypsin and centrifuged to obtain cell pellets. The cell pellets were resuspended in a culture medium and then inoculated onto a 6-well plate at a density of approximately 5×10⁶ cells per well, followed by addition of 2 mL of complete medium and further incubation for 24 hours. An original medium was completely aspirated, and the cells were washed with phosphate-buffered saline (PBS). Then, a fresh medium containing 5 μg/mL puromycin was added. Simultaneously, a lentivirus suspension (multiplicity of infection (MOI)=100) was also added to the 6-well plate, followed by further incubation for 24 hours. The original medium was completely aspirated, and the cells were washed with the PBS. Then, the cells were further cultured for 3-5 days in fresh McCoy's 5a medium containing 10% fetal bovine serum (FBS), screening and identification of an Mlh1 low-expression cell line were performed, ultimately obtaining an Mlh1 low-expression MFC cell line.

b. Construction of a Humanized MSS Mouse Tumor Model

First, a humanized peripheral blood mononuclear cell (Hu-PBMC) model, also referred to as a humanized peripheral blood lymphocyte (Hu-PBL) model, which is a humanized immune system mouse model, was constructed. Six-week-old M-NSG mice (non-obese diabetic, Cg-PrkdcscidIL2rgtm1Wjl/Sz) were selected as transplant recipients. An inoculation dose of peripheral blood mononuclear cells (PBMCs) was 10×10⁶ cells per mouse. Twelve weeks after PBMC transplantation, an implantation level of hCD45⁺ cells was quantitatively determined by flow cytometry of peripheral blood hCD45⁺ cells. Hu-PBMC mice with more than 25% hCD45⁺ cells in peripheral blood were considered successfully transplanted and humanized.

The previously constructed Mlh1 low-expression MFC cell line was collected. After centrifugation and resuspension, cell counting was performed; then, 150 μL of a cell suspension (a cell density of 1× 10⁷ cells/mL) was injected subcutaneously into a right back of each Hu-PBMC mouse.

Tumor-bearing mice were randomly divided into four groups: a normal saline group, a low-dose drug group (20 mg/kg), a medium-dose drug group (40 mg/kg), and a high-dose drug group (80 mg/kg). On day 18 after treatment, the mice were sacrificed, and MFC tissues were collected for photographic analysis.

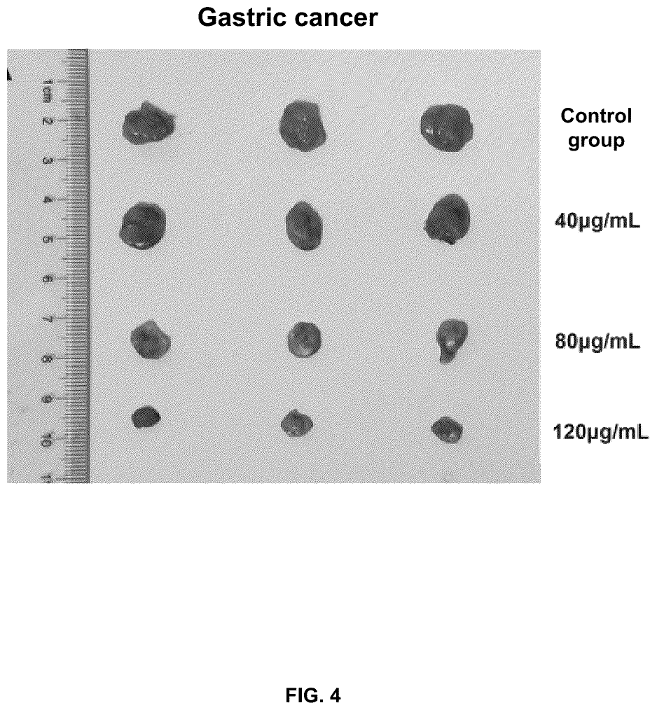

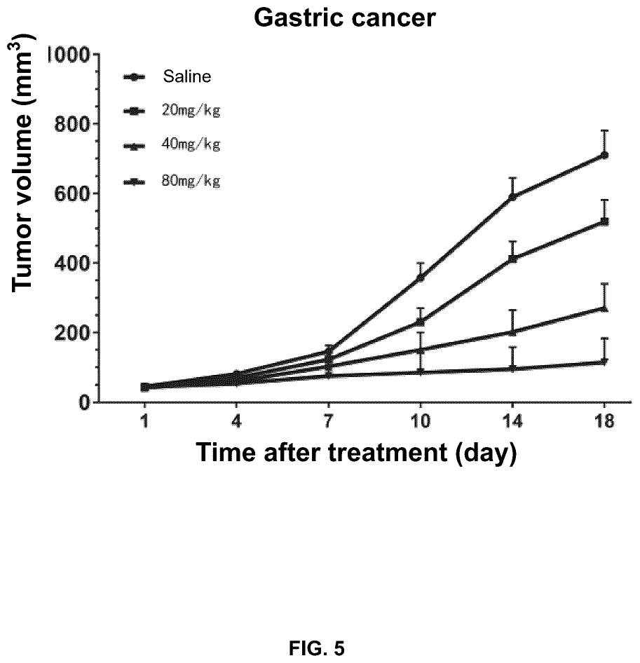

As shown in FIG. 4 and FIG. 5, the icariside I can significantly inhibit tumor growth in vivo and a gastric cancer inhibition rate reaches 80% at a high dose (80 mg/kg).

### Example 4

### Effect of Icariside I Intervention at Different Doses on the Immune System in Gastric Cancer-Bearing Mice In Vivo

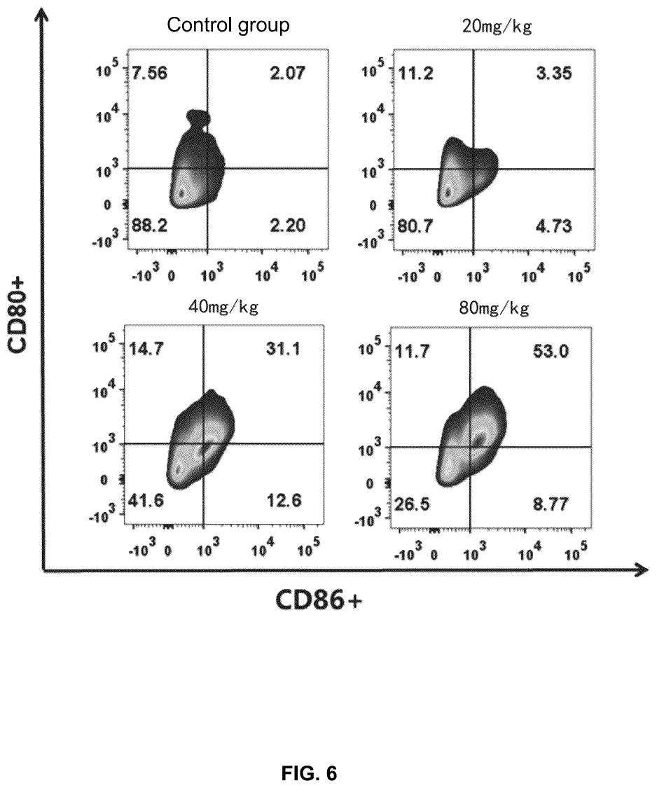

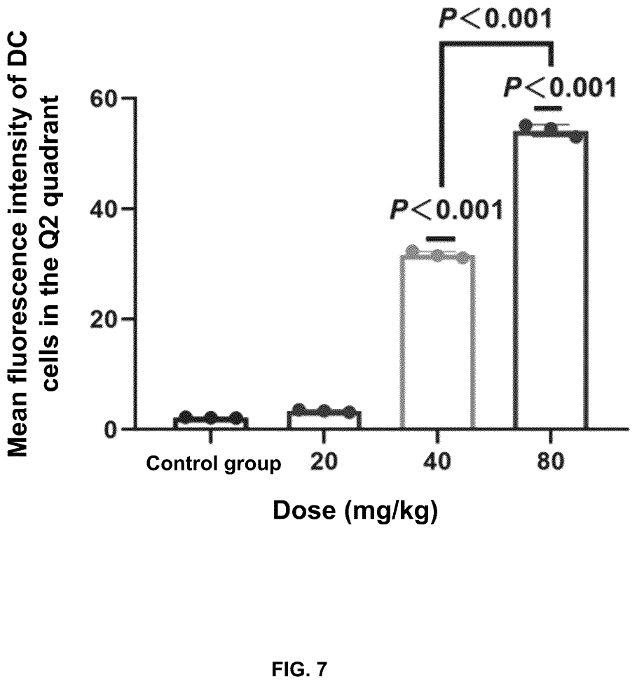

MFC cells were inoculated into balb/c mice to construct subcutaneous gastric cancer-bearing balb/c mouse models. The gastric cancer mouse model was randomly divided into four groups, namely a control group, a low-dose group (20 mg/kg), a medium-dose group (40 mg/kg), and a high-dose group (80 mg/kg), with three mice in each group, which were separately caged and labeled. Daily intragastric administration was performed for 7 consecutive days, and the appetite, defecation, urination, and activity of the mice were observed daily. After 7 days of administration, lymph node tissues from the above four groups were collected, thoroughly minced and ground, and then PBS and trypsin were added. The mixture was repeatedly pipetted to form a single-cell suspension as much as possible. Then, the single-cell suspension was incubated with specific antibodies and then subjected to flow sorting. Flow cytometry was used to analyze immune cells in MFC tissues of mice. The collected single-cell suspension of lymph node tissue was co-incubated with anti-CD80-cy5.5, anti-CD86-FITC, and anti-CD11c-PE flow antibodies. The flow cytometry was used to determine a content of mature dendritic cells (DCs) in the lymph node tissue, and the results are shown in FIG. 6 to FIG. 7.

Experimental results: As shown in FIG. 6 to FIG. 7, intragastric administration of the icariside I at different doses has varying effects on immune cells in the MFC mice. The results indicate that, compared with the gastric cancer mouse models without the icariside I intervention, a count of mature DCs (CD80⁺CD86⁺) in the lymph node tissue after high-dose icariside I intervention is significantly increased, while almost no mature DCs (CD80⁺CD86⁺) are observed in cancer tissues of non-intervention group mice. This indicates that the icariside I can significantly promote maturation of DCs in lymph nodes.

### Example 5

Inhibitory Effect of Combination Therapy with Icariside I and Nivolumab (Hereinafter Referred to as A-PD-1) on Tumor Growth of MSS Gastric Cancer

Nivolumab injection (trade name: Opdivo, Bristol-Myers Squibb, USA) is a fully human monoclonal antibody against a PD-1 receptor. To further verify efficacy of icariside I in enhancing immunotherapeutic effects against gastric cancer, subcutaneous gastric cancer-bearing Hu-PBMC mouse models (MSS) were successfully constructed. The construction of the models was as follows:

a. Construction of a Lentivirus-Transfected MFC Cell Line

The MFC cell line in the logarithmic growth phase was selected, digested with trypsin, and centrifuged to obtain cell pellets. The cell pellets were resuspended in medium and seeded in a 6-well plate, with approximately 5×10⁶ cells per well, followed by addition of 2 mL of complete medium and further incubation for 24 hours. The original medium was completely aspirated, and after washing with PBS, fresh medium containing 5 μg/mL puromycin was added, and a lentiviral suspension (MOI=100) was simultaneously added to the 6-well plate, followed by further incubation for 24 hours. The original medium was completely aspirated, and after washing with the PBS, the cells were further cultured for 3-5 days in the fresh McCoy's 5a medium containing 10% fetal bovine serum (FBS), and screening and identification of the Mlh1 low-expression cell line were performed, finally obtaining the Mlh1 low-expression MFC cell line.

b. Construction of a Humanized MSS Mouse Tumor Model

First, a Hu-PBMC model, also referred to as a Hu-PBL model, which is a humanized immune system mouse model, was constructed. Six-week-old M-NSG mice (non-obese diabetic, Cg-PrkdcscidIL2rgtm1Wjl/Sz) were selected as transplant recipients, and an inoculation dose of PBMCs was 10×10⁶ cells per mouse. Twelve weeks after PBMC transplantation, an implantation level of hCD45 cells was quantitatively determined by flow cytometry of peripheral blood hCD45 cells. Hu-PBMC mice with more than 25% hCD45 cells in peripheral blood were considered to be successfully transplanted and humanized.

The previously constructed Mlh1 low-expression MFC cell line was collected, centrifuged, resuspended, and then subjected to cell counting. Then, 150 μL of cell suspension (a cell density of 1×10⁷ cells/mL) was injected subcutaneously into right back of the mice to construct a MSS mouse tumor model.

The MSS tumor model mice were randomly divided into four groups (namely a saline group, an icariside I 30 mg/kg group, a saline+A-PD-1 group, and an icariside I 30 mg/kg+A-PD-1 group). For the icariside I 30 mg/kg group: the icariside I (30 mg/kg) was administered by daily intragastric administration for 7 consecutive days. For the saline+A-PD-1 group: the A-PD-1 (200 μg per mouse per administration) was administered by intraperitoneal injection (ip) on day 1, day 4, and day 7, respectively. For the icariside I 30 mg/kg+A-PD-1 group: the icariside I (30 mg/kg) was administered by daily intragastric administration for 7 consecutive days from day 1 to day 7, and the A-PD-1 (200 μg per mouse per administration) was simultaneously administered by the intraperitoneal injection on day 1, day 4, and day 7. On day 18 after treatment, the mice were sacrificed, and tumor tissues were collected for photographic analysis.

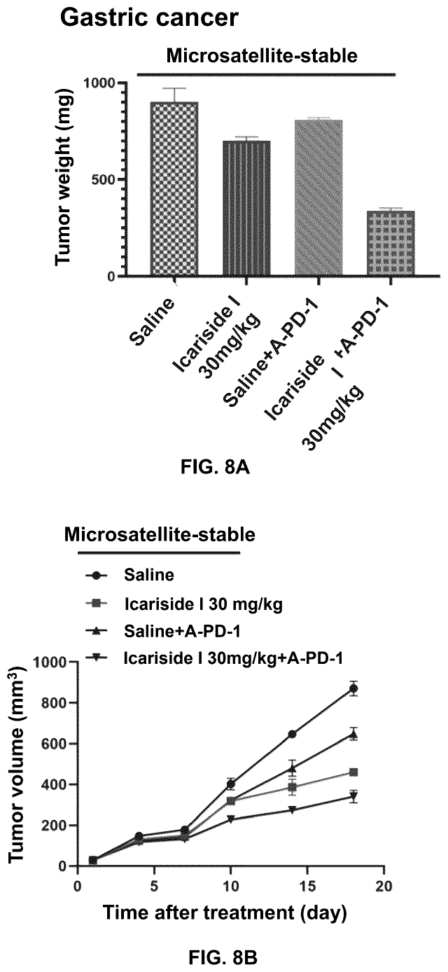

As can be seen from FIG. 8A, in the MSS tumor model, monotherapy with the A-PD-1 exhibits low therapeutic efficacy, a tumor inhibition rate of the icariside I reaches about 30%, whereas a combination of the icariside I and the A-PD-1 results in a tumor inhibition rate as high as 70%. Meanwhile, the tumor growth curves also consistently show that the icariside I combined with the immunotherapeutic agent significantly inhibits the growth of the gastric cancer (FIG. 8B).

### Example 6

Inhibitory Effect of Icariside I Intervention at Different Doses and Combination Therapy with the Icariside I and A-PD-1 on Tumor Growth of MSS Intestinal Cancer

To elucidate an in vivo therapeutic effect of icariside I and an effect of the icariside I on enhancing efficacy of immunotherapy for intestinal cancer, a subcutaneous intestinal cancer-bearing Hu-PBMC mouse model (MSS) was successfully constructed.

The construction of the MSS intestinal cancer mouse model was as follows:

a. Construction of a Lentivirus-Transfected MC38 Intestinal Cancer Cell Line

The MC38 intestinal cancer cell line in the logarithmic growth phase was selected. Using a procedure similar to that for the Mlh1 low-expression MFC cell line, an Mlh1 low-expression MC38 intestinal cancer cell line was successfully constructed.

b. Construction of a Humanized MSS Intestinal Cancer Mouse Model

Using a procedure similar to that for constructing the humanized MSS gastric cancer mouse model, the humanized MSS intestinal cancer mouse model was successfully constructed.

Tumor-bearing mice were randomly divided into five groups, namely a saline group, a low-dose drug group (20 mg/kg), a medium-dose drug group (40 mg/kg), a high-dose drug group (80 mg/kg), and an icariside I+A-PD-1 group (30 mg/kg+A-PD-1). On day 18 after treatment, the mice were sacrificed, and tumor tissues were collected for photographic analysis.

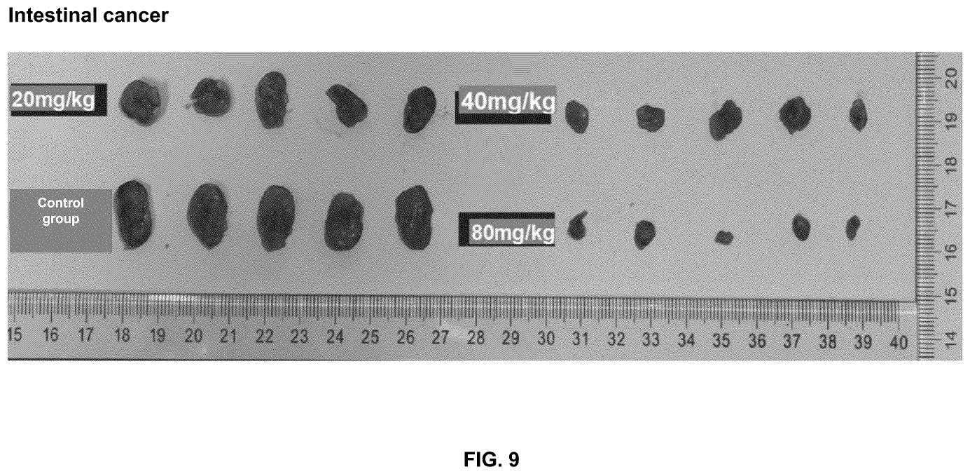

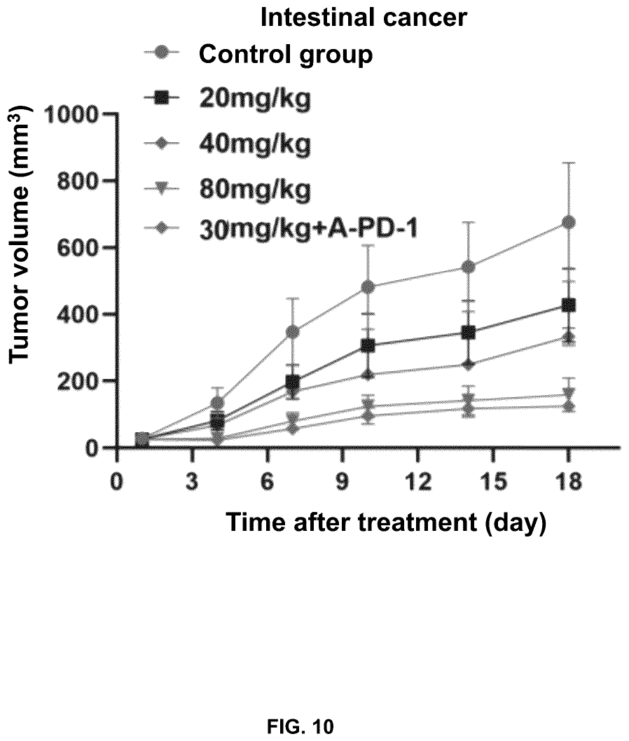

As can be seen from FIG. 9 and FIG. 10, the icariside I can significantly inhibit tumor growth in vivo, with a tumor inhibition rate of up to 76% at a high dose (80 mg/kg). Furthermore, when administered at a dose of 30 mg/kg in combination with an immunotherapeutic agent, the tumor inhibition rate reaches 81%.

### Example 7

### Inhibitory Effect of Icariside I Intervention at Different Doses and Combination Therapy of Icariside I and A-PD-1 on Tumor Growth of MSS Lung Cancer

To elucidate an in vivo therapeutic effect of icariside I and an effect of the icariside I on enhancing efficacy of immunotherapy for lung cancer, a subcutaneous lung cancer-bearing Hu-PBMC mouse model (MSS) was successfully constructed.

The construction of the MSS lung cancer mouse model was as follows:

a. Construction of a Lentivirus-Transfected Lung Cancer Cell Line

The lung cancer cell line in a logarithmic growth phase was selected. Using a procedure similar to that for constructing the Mlh1 low-expression MFC cell line, a Mlh1 low-expression lung cancer cell line was successfully constructed.

b. Construction of a Humanized MSS Lung Cancer Mouse Model

Using a procedure similar to that for constructing the humanized MSS gastric cancer mouse model, a humanized MSS lung cancer mouse model was successfully constructed.

Tumor-bearing mice were randomly divided into five groups: a saline group, a low-dose drug group (20 mg/kg), a medium-dose drug group (40 mg/kg), a high-dose drug group (80 mg/kg), and an icariside I+A-PD-1 group (30 mg/kg+A-PD-1). On day 18 after treatment, the mice were sacrificed, and tumor tissues were collected for photographic analysis.

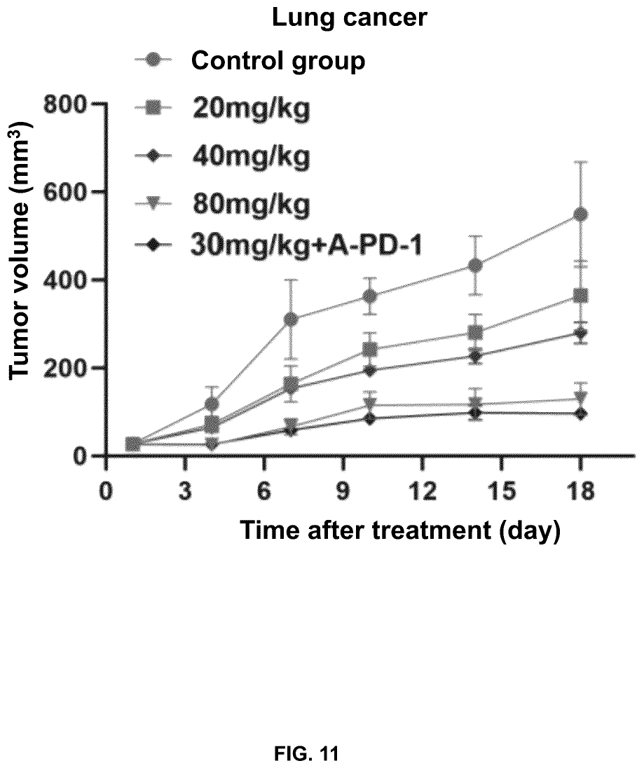

As can be seen from FIG. 11, the icariside I can significantly inhibit tumor growth of lung cancer in vivo, with a tumor inhibition rate reaching 72% at a high dose (80 mg/kg). Moreover, when administered at a dose of 30 mg/kg in combination with an immunotherapeutic agent, the tumor inhibition rate reaches 78%.

### Example 8

### Inhibitory Effect of Icariside I Intervention at Different Doses and Combination Therapy of Icariside I and A-PD-1 on Tumor Growth of MSS Hepatocellular Carcinoma

To elucidate an in vivo therapeutic effect of icariside I and an effect of the icariside I on enhancing an immunotherapeutic effect on hepatocellular carcinoma, a subcutaneous hepatocellular carcinoma-bearing Hu-PBMC mouse model (MSS) was successfully constructed.

Construction of a MSS hepatocellular carcinoma mouse model was as follows:

a. Construction of a Lentivirus-Transfected Hepatocellular Carcinoma Cell Line

The hepatocellular carcinoma cell line in a logarithmic growth phase was selected. Using a procedure similar to that for constructing the Mlh1 low-expression MFC cell line, a Mlh1 low-expression hepatocellular carcinoma cell line was successfully constructed.

b. Construction of a MSS Hepatocellular Carcinoma Mouse Model

Using a procedure similar to that for constructing the humanized MSS gastric cancer mouse model, a humanized MSS hepatocellular carcinoma mouse model was successfully constructed.

Tumor-bearing mice were randomly divided into four groups: a saline group, a low-dose drug group (20 mg/kg), a medium-dose drug group (40 mg/kg), a high-dose drug group (80 mg/kg), and an icariside I+A-PD-1 group (30 mg/kg+A-PD-1). On day 18 after treatment, the mice were sacrificed, and tumor tissues were collected for photographic analysis.

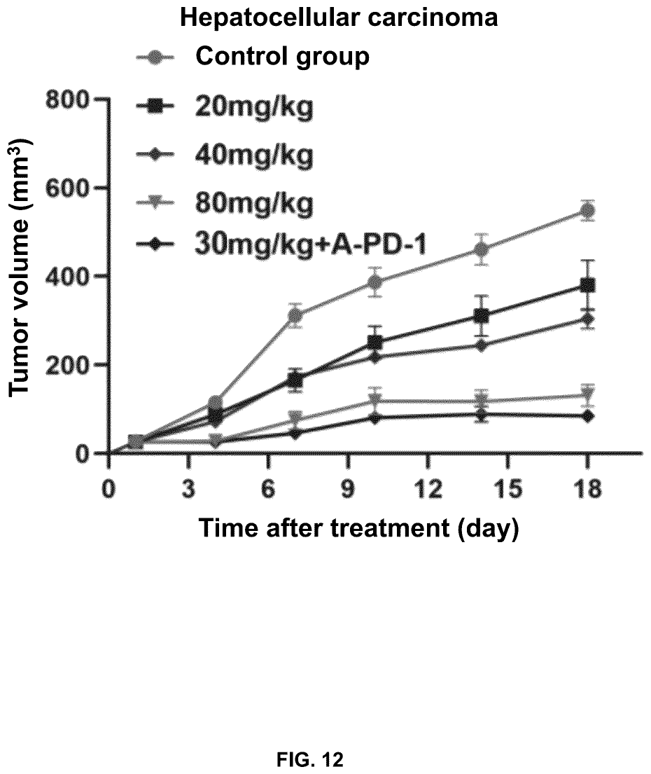

As can be seen from FIG. 12, the icariside I can significantly inhibit tumor growth of hepatocellular carcinoma in vivo, with a tumor inhibition rate reaching 73% at a high dose (80 mg/kg). Moreover, when administered at a dose of 30 mg/kg in combination with an immunotherapeutic agent, the tumor inhibition rate reaches 77%.

### Example 9

### Inhibitory Effect of Icariside I Intervention at Different Doses and Combination Therapy of Icariside I and A-PD-1 on Tumor Growth of MSS Pancreatic Cancer

To elucidate an in vivo therapeutic effect of icariside I and an effect of the icariside I on enhancing an immunotherapeutic effect on pancreatic cancer, a subcutaneous pancreatic cancer-bearing Hu-PBMC mouse model (MSS) was successfully constructed.

Construction of a MSS pancreatic cancer mouse model was as follows:

a. Construction of a Lentivirus-Transfected Pancreatic Cancer Cell Line

The pancreatic cancer cell line in a logarithmic growth phase was selected. Using a procedure similar to that for constructing the Mlh1 low-expression MFC cell line, a Mlh1 low-expression pancreatic cancer cell line was successfully constructed.

b. Construction of a MSS Subcutaneous Pancreatic Cancer Mouse Model

Using a procedure similar to that for constructing the humanized MSS gastric cancer mouse model, a humanized microsatellite-stable pancreatic cancer mouse model was successfully constructed.

Tumor-bearing mice were randomly divided into five groups: a saline group, a low-dose drug group (20 mg/kg), a medium-dose drug group (40 mg/kg), a high-dose drug group (80 mg/kg), and an icariside I+A-PD-1 group (30 mg/kg+A-PD-1). On day 18 after treatment, the mice were sacrificed and tumor tissues were collected for photographic analysis.

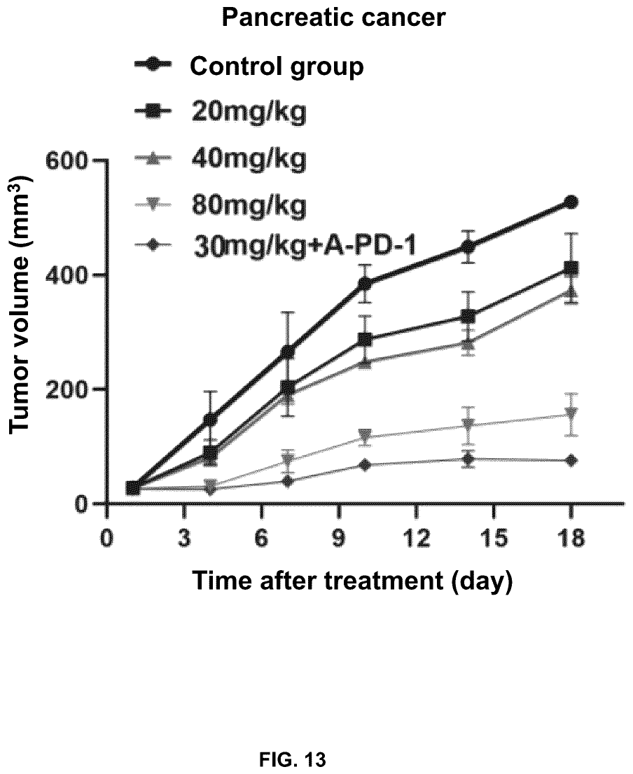

As can be seen from FIG. 13, the icariside I can significantly inhibit tumor growth of pancreatic cancer in vivo, with a tumor inhibition rate reaching 70% at a high dose (80 mg/kg). Moreover, when administered at a dose of 30 mg/kg in combination with an immunotherapeutic agent, the tumor inhibition rate reaches 79%.

### Example 10

### Inhibitory Effect of Icariside I Intervention at Different Doses on Growth of MSS Tumor

To clarify an effective dose range of icariside I, this example set gradient dose groups of the icariside I in tumor-bearing mouse models: 5 mg/kg, 10 mg/kg, 20 mg/kg, 40 mg/kg, 80 mg/kg, 120 mg/kg, and 160 mg/kg.

The tumor-bearing mouse model was the previously constructed MSS mouse model. The tumor-bearing mouse model may be selected from the group consisting of a subcutaneous gastric cancer-bearing BALB/c mouse model, a subcutaneous intestinal cancer-bearing Hu-PBMC mouse model, a subcutaneous lung cancer-bearing Hu-PBMC mouse model, a subcutaneous hepatocellular carcinoma-bearing Hu-PBMC mouse model, and a subcutaneous pancreatic cancer-bearing Hu-PBMC mouse model. More descriptions for constructing the tumor-bearing mouse model may be found in the related descriptions above.

Tumor-bearing mice were randomly divided into eight groups: one saline group (i.e., a control group) and seven gradient dose drug groups (i.e., 5 mg/kg, 10 mg/kg, 20 mg/kg, 40 mg/kg, 80 mg/kg, 120 mg/kg, and 160 mg/kg, respectively). The tumor-bearing mice were administered the icariside I at corresponding group doses by daily intragastric administration for 7 consecutive days. On day 18 after treatment, the mice were sacrificed and tumor tissues were collected for photographic analysis.

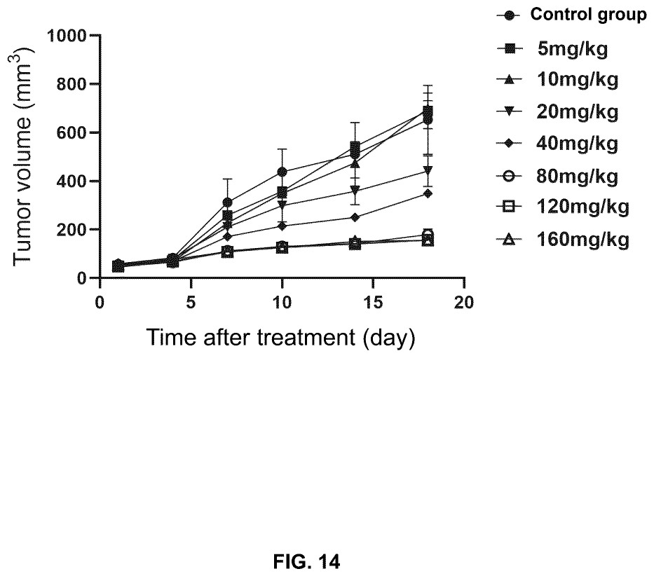

As can be seen from FIG. 14, the icariside I at a dose of 5 mg/kg has a weak inhibitory effect on tumor growth, with a tumor growth curve close to that of the control group. When a dose of the icariside I reaches 10 mg/kg, the tumor growth curve begins to deviate significantly, showing initial tumor-inhibiting activity. This indicates that a lower effective dose limit of the icariside I is about 10 mg/kg. Within a range of 10 mg/kg to 120 mg/kg, a tumor-inhibiting effect gradually increases with an increase in the dose of the icariside I. Tumor volumes of 80 mg/kg and 120 mg/kg dose groups are significantly smaller than tumor volumes of low-dose groups, showing a potent growth inhibitory effect. When a dose of the icariside I increases to 160 mg/kg, no significant difference in tumor inhibition is observed compared to the 120 mg/kg dose group, indicating that a therapeutic effect of the drug enters a plateau phase for the disease condition; therefore, 120 mg/kg may be considered an upper effective dose limit.

### Example 11

### Inhibitory Effect of Icariside I Intervention at Different Doses and Combination Therapy of the Icariside I and a CAR-T Therapy Product on Growth of MSS Tumor

To verify a synergistic effect of the icariside I and a CAR-T therapy product (axicabtagene ciloleucel, targeting CD19, with a structure of 1D3 scFv+CD28+CD3ζ), an experiment set a control group, icariside I monotherapy groups at different doses, and corresponding icariside I+CAR-T groups.

The tumor-bearing mouse model was the previously constructed MSS mouse model. More descriptions for constructing the tumor-bearing mouse model may be found in the related descriptions above.

Tumor-bearing mice were randomly divided into nine groups: one saline group, four icariside I monotherapy groups at different doses (i.e., 5 mg/kg, 10 mg/kg, 120 mg/kg, and 160 mg/kg), and four corresponding icariside I+CAR-T groups (i.e., 5 mg/kg+CAR-T, 10 mg/kg+CAR-T, 120 mg/kg+CAR-T, and 160 mg/kg+CAR-T). For the icariside I monotherapy groups: the tumor-bearing mice were administered the icariside I at corresponding group doses by daily intragastric administration for 7 consecutive days. For the icariside I+CAR-T groups: the tumor-bearing mice were administered the icariside I at corresponding group doses by the daily intragastric administration for 7 consecutive days, simultaneously, the CAR-T therapy product was administered via tail vein injection on day 1, day 4, and day 7. On day 18 after treatment, the mice were sacrificed, and tumor tissues were collected for photographic analysis.

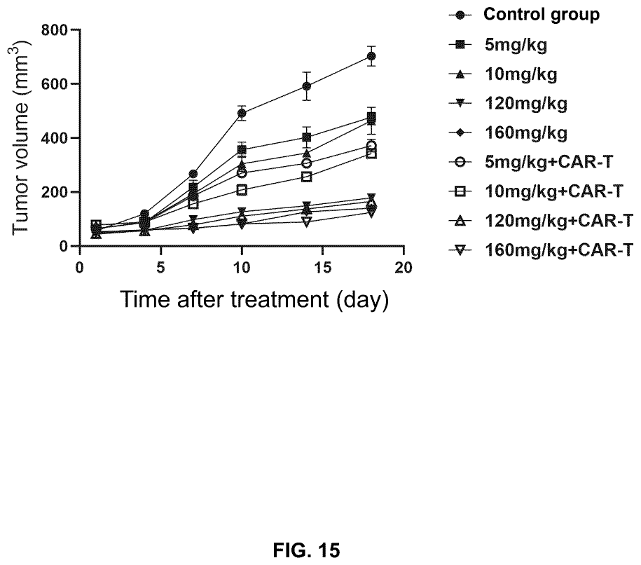

As can be seen from FIG. 15, monotherapy with the icariside I has a limited inhibitory effect on an MSS solid tumor. However, when combined with the CAR-T therapy product, anti-tumor activity of each dose group is improved to varying degrees. For example, an inhibitory effect on tumor growth in a low-dose combination group (e.g., 10 mg/kg+CAR-T) is significantly stronger than that of administration of the icariside I alone at a corresponding dose. In a high-dose combination group (120 mg/kg+CAR-T), the tumor growth is significantly restricted, a tumor volume curve remains at a low level, and an inhibitory effect is also significantly better than that of administration of the icariside I alone at a corresponding dose.

### Example 12

### Inhibitory Effect of Icariside I Intervention at Different Doses and Combination Therapy of the Icariside I and A-PD-1 on Growth of MSS Solid Tumor

To verify the synergistic effect of icariside I and A-PD-1, an experiment set a control group, monotherapy groups of the icariside I at different doses, and corresponding combination groups.

The tumor-bearing mouse model was the previously constructed MSS mouse model. More descriptions of constructing the tumor-bearing mouse model may be found in the related descriptions above.

Tumor-bearing mice were randomly divided into 13 groups: a saline group, six monotherapy groups of the icariside I at different doses (i.e., 5 mg/kg, 20 mg/kg, 40 mg/kg, 80 mg/kg, 120 mg/kg, and 160 mg/kg), and six corresponding icariside I+A-PD-1 groups (i.e., 5 mg/kg+A-PD-1, 20 mg/kg+A-PD-1, 40 mg/kg+A-PD-1, 80 mg/kg+A-PD-1, 120 mg/kg+A-PD-1, and 160 mg/kg+A-PD-1).

For icariside I monotherapy groups: the tumor-bearing mice were administered the icariside I at corresponding doses via daily intragastric administration for 7 consecutive days. For icariside I+A-PD-1 groups: the tumor-bearing mice were administered the icariside I at doses corresponding to the respective groups via the daily intragastric administration for 7 consecutive days and were simultaneously administered the A-PD-1 (200 μg per animal per administration) via intraperitoneal injection on day 1, day 4, and day 7. On day 18 after treatment, the mice were sacrificed, and tumor tissues were collected for photographic analysis.

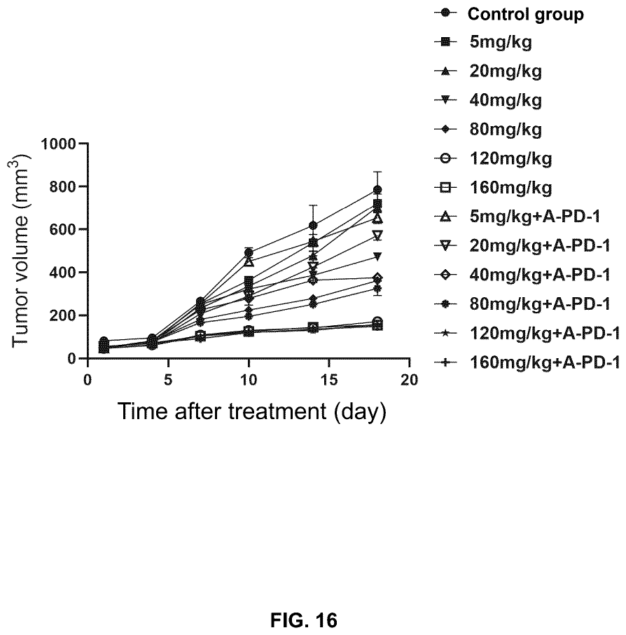

As can be seen from FIG. 16, monotherapy of the icariside I has a limited inhibitory effect on the MSS solid tumor. However, when combined with the A-PD-1, anti-tumor activity of each dose group is improved to varying degrees. In the combination groups of 20 mg/kg+A-PD-1, 40 mg/kg+A-PD-1, 80 mg/kg+A-PD-1, and 120 mg/kg+A-PD-1, an inhibitory effect on tumor growth is significantly better than that of administration of the icariside I alone at the corresponding dose.

### Example 13

Synergistic Therapeutic Effect of Icariside I Combined with A-PD-1 and CAR-T Cells

To verify a synergistic effect of icariside I combined with A-PD-1 and CAR-T cells, an experiment was conducted to assess in vivo anti-tumor activity of the icariside I in a dose range of 5-160 mg/kg in combination with the A-PD-1 and the CAR-T cells (axicabtagene ciloleucel, targeting CD19, containing a structure of 1D3 scFv+CD28+CD3ζ).

The tumor-bearing mouse model was the previously constructed MSS mouse model. More descriptions of constructing the tumor-bearing mouse model may be found in the related descriptions above.

Tumor-bearing mice were randomly divided into 16 groups: a saline group, an A-PD-1+CAR-T treatment group, seven monotherapy groups of the icariside I at different doses (i.e., 5 mg/kg, 10 mg/kg, 20 mg/kg, 40 mg/kg, 80 mg/kg, 120 mg/kg, and 160 mg/kg), and seven corresponding combination groups (i.e., 5 mg/kg+A-PD-1+CAR-T, 10 mg/kg+A-PD-1+CAR-T, 20 mg/kg+A-PD-1+CAR-T, 40 mg/kg+A-PD-1+CAR-T, 80 mg/kg+A-PD-1+CAR-T, 120 mg/kg+A-PD-1+CAR-T, and 160 mg/kg+A-PD-1+CAR-T).

For the icariside I monotherapy groups: the tumor-bearing mice were administered the icariside I at corresponding doses via daily intragastric administration for 7 consecutive days. For the A-PD-1+CAR-T treatment group, the tumor-bearing mice were simultaneously administered the A-PD-1 (200 μg per animal per administration) via intraperitoneal injection on day 1, day 4, and day 7 and the CAR-T cells via intravenous injection on day 1, day 4, and day 7. For the combination groups: the tumor-bearing mice were administered icariside I at corresponding doses via daily intragastric administration for 7 consecutive days, and were simultaneously administered A-PD-1 (200 μg per animal per administration) via intraperitoneal injection on day 1, day 4, and day 7 and CAR-T cells via intravenous injection on day 1, day 4, and day 7. After 18 days of the treatment, the mice were sacrificed, and tumor tissues were collected for photographic analysis.

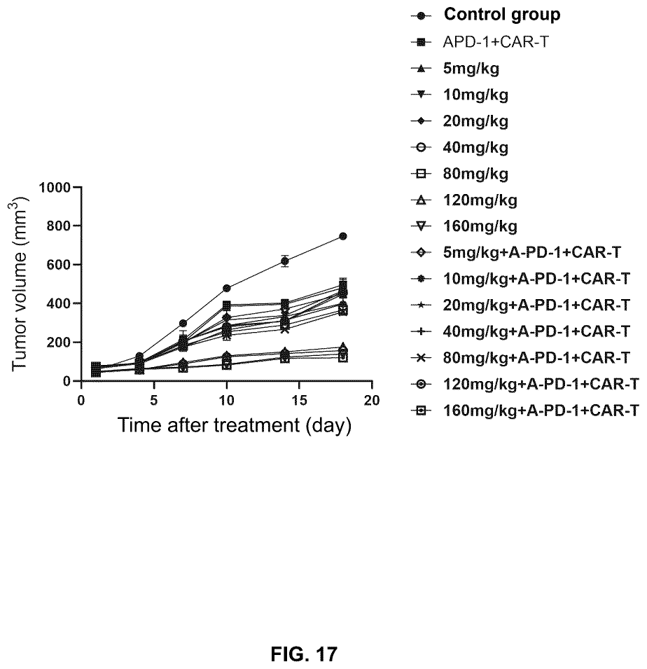

As can be seen from FIG. 17, a tumor volume of the control group increases rapidly over time, while treatment with the icariside I alone or the A-PD-1+CAR-T only exhibits a limited anti-tumor effect. After application of the icariside I combined with the A-PD-1 and the CAR-T cells, all dose groups exhibit sensitizing effects to varying degrees. Within an effective dose range of 20-120 mg/kg, tumor growth is most significantly restricted in the groups treated with the icariside I combined with the A-PD-1 and the CAR-T cells, and tumor volume curves of the groups treated with the icariside I combined with the A-PD-1 and the CAR-T cells are significantly lower than those of the icariside I monotherapy groups and the A-PD-1+CAR-T treatment group. Moreover, when the dose is further increased to 160 mg/kg, the anti-tumor effect reaches a plateau, with no further enhancement observed. This confirms that the specific combination regimen can maximize the reversal of immunosuppressive state of MSS solid tumors while maintaining low systemic toxicity.

As can be seen from the above examples, the icariside I provided by the embodiments of the present disclosure has a clear dose-effect relationship within a range of 10-120 mg/kg. Through combination with the CAR-T and/or the A-PD-1, an anti-tumor ceiling of a single immunotherapy is raised, and the experiment proves that doses above 160 mg/kg do not result in a further significant enhancement of efficacy, thereby defining 120 mg/kg as a reference upper limit for clinical administration.

According to the above examples, the icariside I can be used as a drug for inhibiting the tumor cells and enhancing tumor immunotherapy. Administration of the icariside I in combination with the tumor immunotherapy provides an effective therapeutic effect against the tumors.

The above implementations are merely preferred implementations of some embodiments of the present disclosure, and should not be used to limit the scope protected by some embodiments of the present disclosure. Any non-substantial changes and substitutions made by those skilled in the art based on some embodiments of the present disclosure fall within the scope claimed by some embodiments of the present disclosure.

What is claimed is:

1. A method for treating a microsatellite-stable solid tumor, comprising administering a therapeutically effective amount of icariside I to a subject suffering from the microsatellite-stable solid tumor.

2. The method of claim 1, wherein the microsatellite-stable solid tumor is selected from the group consisting of a microsatellite-stable gastric cancer, a microsatellite-stable intestinal cancer, a microsatellite-stable lung cancer, a microsatellite-stable pancreatic cancer, and a microsatellite-stable hepatocellular carcinoma.

3. The method of claim 1, wherein the icariside I is in a form of a tablet, a capsule, a granule, a softgel, a drop pill, a syrup, an injection, or a loaded nanocomposite.

4. The method of claim 1, further comprising administering a combination anti-cancer active agent to the subject.

5. The method of claim 4, wherein the combination anti-cancer active agent comprises an immunotherapeutic agent.

6. The method of claim 5, wherein the immunotherapeutic agent comprises at least one of a PD-1/PD-L1 inhibitor or a CAR-T therapy product.

7. The method of claim 6, wherein the PD-1/PD-L1 inhibitor comprises at least one of nivolumab, sintilimab, camrelizumab, atezolizumab, or pembrolizumab.

8. The method of claim 6, wherein the CAR-T therapy product comprises at least one of axicabtagene ciloleucel or relmacabtagene autoleucel.

9. The method of claim 1, wherein the therapeutically effective amount of the icariside I is 10-120 mg per kg body mass of the subject (mg/kg).

10. The method of claim 1, wherein the therapeutically effective amount of the icariside I is 20-80 mg/kg.

11. The method of claim 1, further comprising: administering a combination anti-cancer active agent comprising an immunotherapeutic agent to the subject, wherein the therapeutically effective amount of the icariside I is 30 mg/kg.

12. The method of claim 1, wherein the administering the icariside I comprises: administering the icariside I once daily by intragastric administration or oral administration for 7 consecutive days.

13. The method of claim 12, further comprising: injecting an immunotherapeutic agent on day 1, day 4, and day 7.

14. The method of claim 13, wherein the immunotherapeutic agent is nivolumab.

15. The method of claim 13, wherein the immunotherapeutic agent is axicabtagene ciloleucel.

16. The method of claim 12, further comprising: administering nivolumab and axicabtagene ciloleucel to the subject.

17. A kit for treating a microsatellite-stable solid tumor, wherein the kit comprises icariside I.

18. The kit of claim 17, wherein the kit further comprises an immunotherapeutic agent.

19. The kit of claim 18, wherein the immunotherapeutic agent comprises at least one of a PD-1/PD-L1 inhibitor or a CAR-T therapy product.

20. The kit of claim 17, wherein the microsatellite-stable solid tumor is selected from the group consisting of a microsatellite-stable gastric cancer, a microsatellite-stable intestinal cancer, a microsatellite-stable lung cancer, a microsatellite-stable pancreatic cancer, and a microsatellite-stable hepatocellular carcinoma.
